(19) United States
(12) Patent Application Publication
(10) Pub. No.: US 2026/0354824 A1
(43) Pub. Date: Nov. 12, 2026

(54) ARTICULATION LOCK ACTUATABLE BY A SURGICAL FUNCTION TO RESIST DE-ARTICULATION

(71) Applicant: Cilag GmbH International, Zug (CH)

(72) Inventors: Weston Stanley Hirschfeld, West Chester, OH (US); Brett Andrew Rich, Crestview Hills, KY (US); Michael Steven Bahan, Liberty Township, OH (US); Jason E. Zerkle, Blanchester, OH (US); Nicholas David Courtwright, Villa Hills, KY (US); Thomas Allen Stoughton, Cincinnati, OH (US); Brendan J. Oberkircher, Cincinnati, OH (US)

(73) Assignee: Cilag GmbH International, Zug (CH)

(21) Appl. No.: 19/201,506

(22) Filed: May 7, 2025

(51) Int. Cl. A61B 17/072; A61B 17/00; A61B 90/00

(52) U.S. Cl. CPC A61B 17/07207; A61B 90/03; A61B 2017/00477; A61B 2090/035

(57) ABSTRACT

A surgical device is disclosed. The surgical device may include an elongate shaft, an end effector, an articulation joint, an articulation band, an actuator, and an articulation lock. The end effector may be rotatable relative to the elongate shaft at the articulation joint through an articulation motion between unarticulated and articulated configurations. The articulation band may extend distally to the articulation joint. The articulation band may be translatable to rotate the end effector through the articulation motion. The actuator may be actuatable to transmit a force across the articulation joint to the end effector to effect a surgical function. The articulation lock may be structured to lock the articulation joint in the articulated configuration. The articulation lock may be movable between an unlocked and locked position. Actuation of the actuator may be structured and positioned to move the articulation lock from the unlocked position to the locked position.

### BACKGROUND

This disclosure generally relates to surgical tools and, more particularly, to articulation locks for surgical tools.

A surgical tool may be an articulatable surgical tool having an end effector that is articulatable, or rotatable, relative to a shaft of the surgical tool. The articulatable end effector may be configurable in an orientation to reach tissue that is otherwise inaccessible or difficult to reach with a non-articulatable end effector. An articulatable surgical tool may include an articulation joint or wrist at which the end effector is rotatable relative to the shaft. An articulatable surgical tool may include an articulation lock to hold the end effector at a predefined articulation angle relative to the shaft. Exemplary articulatable surgical tools are described in, for example, U.S. Pat. No. 5,713,505, titled ARTICULATION TRANSMISSION MECHANISM FOR SURGICAL INSTRUMENTS, which issued Feb. 3, 1998; U.S. Pat. No. 9,662,110, titled SURGICAL STAPLING INSTRUMENT WITH AN ARTICULATABLE END EFFECTOR, which issued May 30, 2017; and U.S. Pat. No. 11,234,696, titled ARTICULATION DRIVE FEATURES FOR SURGICAL STAPLER, which issued Feb. 1, 2022.

### SUMMARY

One general aspect includes a surgical device. The surgical device may include an elongate shaft, an end effector, an articulation joint, an articulation band, an actuator, and an articulation lock. The end effector may be rotatable relative to the elongate shaft at the articulation joint through an articulation motion between an unarticulated configuration and an articulated configuration. The articulation band may extend distally to the articulation joint, and the articulation band may be translatable to rotate the end effector through the articulation motion. The actuator may be actuatable to transmit a force across the articulation joint to the end effector to effect a surgical function. The articulation lock may be structured to lock the articulation joint in the articulated configuration, the articulation lock may be movable between an unlocked position and a locked position, and actuation of the actuator may be structured and positioned to move the articulation lock from the unlocked position to the locked position.

Implementations of the surgical device may include one or more of the following features.

The surgical function may include staple firing. The articulation lock may include an articulation plate and a locking tab. The articulation plate may be rotatable about an axis to rotate the end effector through the articulation motion based on translation of the articulation band. The articulation plate may include at least one slot positioned along a perimeter of the articulation plate, and, in the locked position, the locking tab may be positionable in the at least one slot to prevent rotation of the end effector relative to the elongate shaft. The articulation lock may include a spring to bias the locking tab toward the unlocked position. The actuator may include a firing bar, the firing bar may include a recess, and, in a first position of the firing bar corresponding to the unlocked position of the articulation lock, the recess may be sized and positioned to receive the locking tab. The recess may include at least one cam, and actuation of the firing bar may move the at least one cam along the locking tab to cam the locking tab into locking engagement with the at least one slot. After the at least one cam cams the locking tab into locking engagement with the at least one slot, a surface of the firing bar may maintain the locking tab in the locked position during actuation of the firing bar. The locked position may be defined by a meshing engagement between the articulation plate and the locking tab, and the unlocked position may be defined by a gap between the articulation plate and the locking tab. The articulation plate may include a first articulation plate and a second articulation plate. The first articulation plate may be coupled to the articulation band, the second articulation plate may be coupled to the end effector, the first articulation plate may be rotatably coupled to the second articulation plate, and the locking tab may meshingly engage the first articulation plate and the second articulation plate in the locked position of the articulation lock.

The surgical function may include jaw closure. The actuator may include a closure tube, the articulation lock may include a compressible clamp positioned laterally between the closure tube and the articulation band, the closure tube may include a protrusion extending laterally inward toward the spring, and, in the locked position, the protrusion may be positionable to apply a compressive force to the compressible clamp. The protrusion may include a stamped indent in the closure tube. The locked position may be defined by a locking contact between the protrusion and the compressible clamp, and the unlocked position may be defined by a gap between the protrusion and the compressible clamp. In the locked position, a frictional force between the compressible clamp and the articulation band may prevent translation of the articulation band, thereby preventing rotation of the end effector through the articulation motion.

The articulation lock may be a secondary lock, and the surgical device may include a primary articulation lock. The primary articulation lock may be a passive articulation lock to resist de-articulation forces up to a first threshold, and the secondary articulation lock may be an active articulation lock to resist de-articulation forces up to a second threshold. The second threshold may be greater than the first threshold.

The articulation band may include a plurality of articulation bands to push and pull the articulation joint through the articulation motion. The actuator may include at least one of a closure trigger or a firing trigger.

Another general aspect includes a surgical device. The surgical device may include an elongate shaft, an end effector, an articulation joint, an actuator, and an articulation lock. The end effector may be rotatable relative to the elongate shaft at the articulation joint through an articulation motion between an unarticulated configuration and an articulated configuration. The actuator may be actuatable to transmit a force to the end effector to effect a surgical function. The articulation lock may be structured to lock the articulation joint, the articulation lock may be movable between an unlocked position and a locked position, and an actuation of the actuator may be structured and positioned to move the articulation lock from the unlocked position to the locked position.

Another general aspect includes a surgical device. The surgical device may include an end effector, an articulation joint, an actuator, and an articulation lock. The end effector may be articulatable about an axis of the articulation joint between an unarticulated configuration and an articulated configuration. The actuator may be actuatable to transmit a force to the end effector to effect a surgical function. The articulation lock may be structured to lock the articulation joint, the articulation lock may be movable between an unlocked position and a locked position, and an actuation of the actuator may be structured and positioned to move the articulation lock from the unlocked position to the locked position.

### BRIEF DESCRIPTION OF THE DRAWINGS

The accompanying drawings, where like reference numerals may refer to functionally similar elements throughout the separate views, together with the detailed description below, are incorporated in and form part of the specification, and serve to further illustrate aspects of concepts that include the claimed disclosure and explain various principles and advantages of those aspects.

FIG. 1 is a perspective view of a surgical device, according to at least one aspect of the present disclosure.

FIG. 2 is a perspective view of a proximal end of a shaft assembly with a proximal housing removed for illustrative purposes, according to at least one aspect of the present disclosure.

FIG. 3 is a plan view of the proximal end of the shaft assembly of FIG. 2 with an articulation control knob removed for illustrative purposes, according to at least one aspect of the present disclosure.

FIG. 4 is a plan view of a distal end of a shaft assembly with a portion of the closure tube removed for illustrative purposes, according to at least one aspect of the present disclosure.

FIG. 5 is an exploded perspective view of the distal end of the shaft assembly of FIG. 4, according to at least one aspect of the present disclosure.

FIG. 6 is a cross-sectional view of a portion of a shaft assembly, illustrating an articulation lock in an unlocked position, according to at least one aspect of the present disclosure.

FIG. 7 is an enlarged cross-sectional perspective view of the articulation lock of FIG. 6 in the unlocked position, according to at least one aspect of the present disclosure.

FIG. 8 is a cross-sectional view of a portion of a shaft assembly, illustrating an articulation lock in a locked position, according to at least one aspect of the present disclosure.

FIG. 9 is an enlarged cross-sectional perspective view of the articulation lock of FIG. 8 in the locked position, according to at least one aspect of the present disclosure.

FIG. 10 is a perspective view of a portion of a shaft assembly with a portion of a closure tube removed for illustrative purposes, illustrating an articulation lock in an unlocked position, according to at least one aspect of the present disclosure.

FIG. 10A is an enlarged perspective view of the articulation lock of FIG. 10 in the unlocked position, according to at least one aspect of the present disclosure.

FIG. 11 is a perspective view of a portion of a shaft assembly foreshortened along an elongated shaft thereof and with a portion of a closure tube removed for illustrative purposes, illustrating an articulation lock in a locked position, according to at least one aspect of the present disclosure.

FIG. 11A is an enlarged perspective view of the articulation lock of FIG. 11 in the locked position, according to at least one aspect of the present disclosure.

The surgical tools and articulation locks disclosed herein have been represented where appropriate by conventional symbols in the drawings, showing only those specific details that are pertinent to understanding the various aspects of the present disclosure so as not to obscure the disclosure with details that will be readily apparent to those of ordinary skill in the art having the benefit of the description herein.

### DETAILED DESCRIPTION

The present disclosure is directed to surgical tools having an articulation lock. In the description, for purposes of explanation and not limitation, specific details are set forth, such as particular aspects, procedures, techniques, etc. to provide a thorough understanding of the present technology. However, it will be apparent to one skilled in the art that the present technology may be practiced in other aspects that depart from these specific details.

Articulatable surgical tools may incorporate different types of locks for holding one or more articulatable components, such as an end effector, for example, in an articulated configuration. For example, the end effector can be locked at a set articulation angle relative to the shaft and the articulation angle can be maintained through a surgical function, such as during a clamping, stapling, and/or cutting motion, for example, until the articulation lock is released. Articulation locks may facilitate controlled positioning of the articulatable surgical tool during a surgical procedure, which may improve a user's ability to manipulate tissue and/or perform other actions with precision and stability, for example.

Articulation locks may include passive locks, which may engage and lock an articulated position of an end effector automatically, or active locks, which may require a user input to engage and lock an articulated position of the end effector. In various instances, active articulation locks may be employed to engage and lock an articulated position of the end effector based on a surgical function, such as a closing motion or a firing motion. In various instances, active articulation locks may be employed in addition to or in lieu of passive articulation locks to prevent de-articulation of the end effector from an articulated configuration toward an unarticulated configuration.

An active articulation lock can be self-locking or self-actuated based on actuation of another surgical function, such as a jaw closure or a staple firing motion. For example, a jaw closure and/or staple firing motion can automatically move the articulation lock into the locked configuration. In various aspects, the articulation lock can automatically return to the unlocked configuration upon completion of the jaw closure and/or staple firing motion and/or retraction of the actuator and/or driver (e.g. a closure tube and/or a firing bar) effecting the surgical function.

In various aspects, the active articulation lock can be actuated at a distal location, which can be at and/or adjacent to the articulation joint. For example, the articulation lock can be a distal articulation lock positioned to interact with the articulation joint to actively resist de-articulation thereof at the articulation joint throughout a surgical function.

The ability to actively and reliably lock the end effector in an articulated configuration can be important in procedures requiring precise angular positioning relative to tissue to be clamped, stapled, and/or cut. For example, unintentional de-articulation may compromise procedural outcomes or result in damage to surrounding tissue.

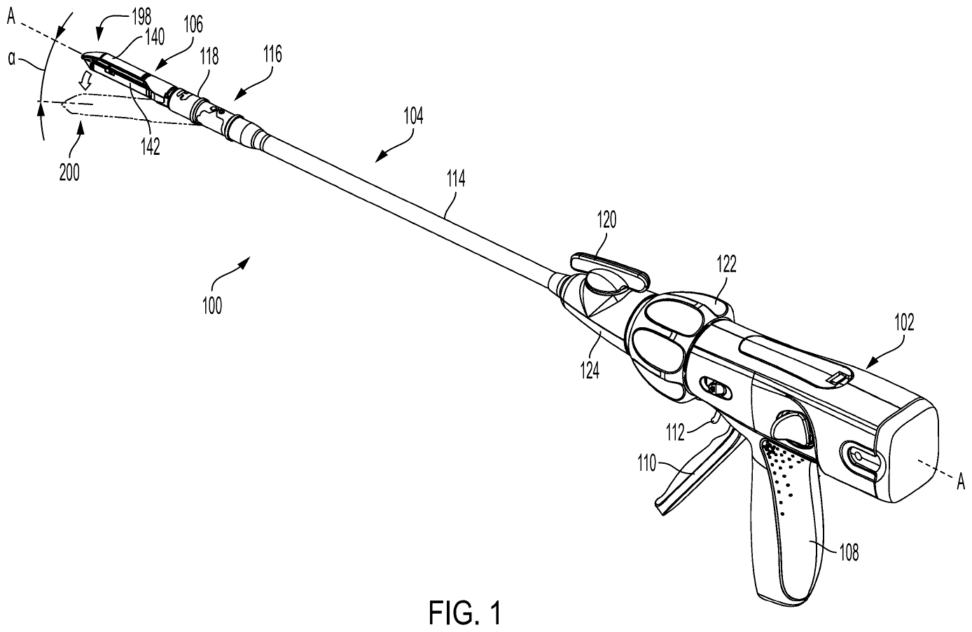

Referring primarily to FIG. 1, a surgical device 100 is disclosed. The surgical device 100 includes a handle assembly 102, a shaft assembly, or an elongate shaft, 104, and an end effector 106. The handle assembly 102 includes a handle 108, a closure trigger 110, and a firing trigger 112. The handle 108 enables a user to grip and manipulate the surgical device 100. The closure trigger 110 and the firing trigger 112 are selectively pivotable toward and away from the handle 108, as further described herein. In various instances, the handle assembly 102 may include other features in addition to or in lieu of the features described herein. In various instances, the surgical device 100 may include any suitable assembly in addition to or in lieu of the handle assembly 102, including but not limited to any suitable assembly configured for use with a robotic surgical system. For example, the surgical device 100 can be a robotic surgical tool and, in certain instances, the triggers and/or actuators therefor may be remote to the surgical device 100.

The shaft assembly 104 includes a closure tube 114, an articulation joint 116, and a closure ring 118. The closure tube 114 extends along a length of the shaft assembly 104. The articulation joint 116 is positioned distal to the closure tube 114, and the closure ring 118 is positioned distal to the articulation joint 116. The closure tube 114 and the closure ring 118 are configured to translate longitudinally relative to the handle assembly 102. A longitudinal translation of the closure tube 114 is transmitted to the closure ring 118 through the articulation joint 116, regardless of whether the articulation joint 116 is in an unarticulated configuration 198 or an articulated configuration 200. Various suitable features that may be used to provide longitudinal translation of the closure tube 114 and the closure ring 118 will be apparent to those of ordinary skill in the art in view of the present disclosure.

As a user inserts the surgical device 100 into a surgical site, it may be desirable to approach tissue to be clamped, stapled, and/or cut from a particular angle. In various instances, once the end effector 106 of the surgical device 100 is inserted through a trocar, it may be desirable for portions of the surgical device 100, such as the end effector 106, for example, to articulate such that the user may precisely position the end effector 106 relative to the target tissue to clamp, staple, and/or cut the target tissue.

In various instances, the articulation joint 116 is operable to pivot a distal portion of the device 100, such as the closure ring 118 and the end effector 106, for example, relative to the shaft assembly 104, and specifically relative to a longitudinal axis A-A defined by the shaft assembly 104. The closure ring 118 and the end effector 106 may pivot at a desired angle a relative to the longitudinal axis A-A. In various instances, the articulation joint 116 may enable articulation about a single articulation axis. In other instances, the articulation joint 116 may enable articulation about more than one articulation axis. The at least one articulation axis can be oriented perpendicular to the longitudinal axis A-A. Rotation of the end effector 106 about an articulation axis can be referred to as end effector articulation, for example.

In various instances, articulation is controlled by an articulation control knob 120, which is located at a proximal end of the shaft assembly 104. The articulation control knob 120 is rotatable about an axis that is perpendicular to the longitudinal axis A-A. In response to a rotation of the articulation control knob 120, the closure ring 118 and the end effector 106 are pivotable about an axis that is also perpendicular to the longitudinal axis A-A. In various instances, clockwise rotation of the articulation control knob 120 pivots the closure ring 118 and the end effector 106 in a clockwise direction. In various instances, counterclockwise rotation of the articulation control knob 120 pivots the closure ring 118 and the end effector in a counterclockwise direction.

In other aspects of the present disclosure, the surgical device 100 may not include an articulation control knob 120. For example, the articulation joint 116 may be a passive articulation joint, which is actuated by applying a force at the end effector 106, such as by pushing the end effector 106 against patient tissue to articulate the end effector 116 at the joint. Exemplary passive articulation joints are further disclosed in, for example, U.S. Pat. No. 9,662,110, titled SURGICAL STAPLING INSTRUMENT WITH AN ARTICULATABLE END EFFECTOR, which issued May 30, 2017, and is incorporated by reference herein in its entirety.

Exemplary articulation joints and/or articulation control knobs are described in, for example, U.S. Pat. No. 9,186,142, titled SURGICAL INSTRUMENT END EFFECTOR ARTICULATION DRIVE WITH PINION AND OPPOSING RACKS, which issued Nov. 17, 2015; U.S. Pat. No. 9,795,379, titled SURGICAL INSTRUMENT WITH MULTI-DIAMETER SHAFT, which issued Oct. 24, 2017; and U.S. Pat. No. 10,064,620, titled METHOD OF UNLOCKING ARTICULATION JOINT IN SURGICAL STAPLER, which issued Sep. 4, 2018, each of which is hereby incorporated by reference herein in its respective entirety.

In addition to articulation of the closure ring 118 and the end effector 106 as described herein, as a user inserts the surgical device 100 into a surgical site, it may be desirable to approach tissue to be clamped, stapled, and/or cut from a particular orientation. In various instances, once the end effector 106 of the surgical device 100 is inserted through a trocar it may be desirable for portions of the surgical device 100, such as the end effector 106, for example, to rotate relative to the handle assembly 102 about the longitudinal axis A-A such that the user may precisely position the end effector 106 relative to the target tissue to clamp, staple, and/or cut the target tissue. Rotation of the shaft assembly 104 and the end effector 106 about the longitudinal axis A-A can be referred to as shaft roll, for example.

Referring still to FIG. 1, the shaft assembly 104, and specifically the proximal end of the shaft assembly 104, further includes a rotation control knob 122 which is operable to rotate the shaft assembly 104 about the longitudinal axis A-A. The rotation control knob 122 may be used to rotate portions of the surgical device 100, such as the shaft assembly 104 and the end effector 106, for example, relative to the handle assembly 102, thereby allowing a user to orient the end effector 106 relative to the target tissue without repositioning themselves or rotating the handle assembly 102.

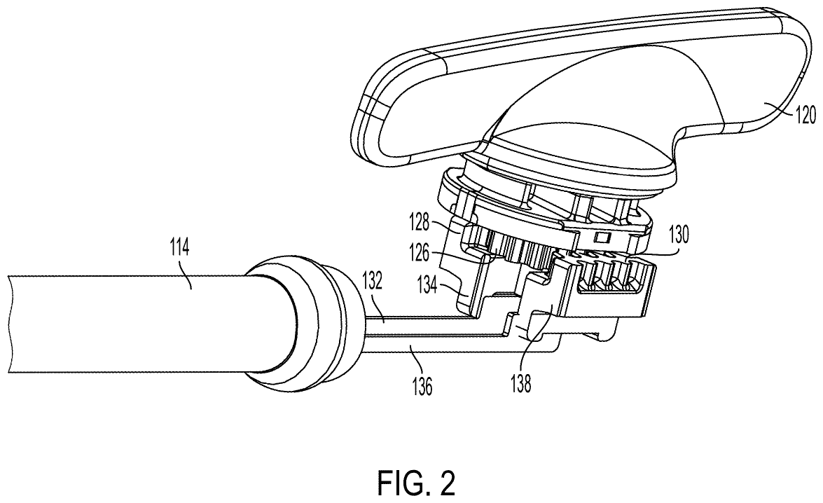

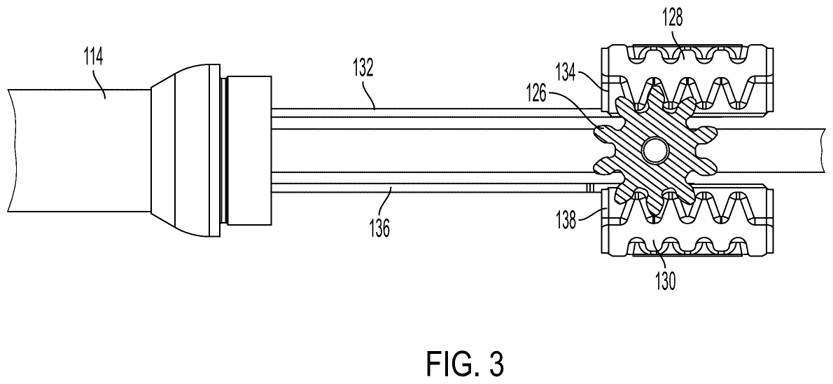

Referring now to FIGS. 2 and 3, the proximal end of the shaft assembly 104 with a proximal housing 124 removed is disclosed. As shown, the articulation control knob 120 is coupled to an articulation pinion 126, which is coupled to a first rack 128 and a second rack 130. The first rack 128 is coupled to a first articulation band 132 by a first intermediate block 134, and the second rack 130 is coupled to a second articulation band 136 by a second intermediate block 138. As a user rotates the articulation control knob 120, the articulation pinion 126 rotates together with the articulation control knob 120. As the articulation control knob 120 rotates the articulation pinion 126, the articulation pinion 126 translates the first rack 128 and the second rack 130 in opposing directions. For example, as the first rack 128 is moved proximally, the second rack 130 is moved distally, and vice versa.

In various instances, as the articulation control knob 120, and thus the articulation pinion 126, rotates clockwise, the first rack 128 retracts proximally away from the end effector 106 and the second rack 130 advances distally toward the end effector 106. As the first rack 128 retracts proximally away from the end effector 106, the first articulation band 132 similarly retracts proximally away from the end effector 106. As the second rack 130 advances distally toward the end effector 106, the second articulation band 136 similarly advances distally toward the end effector 106.

In various instances, as the articulation control knob 120, and thus the articulation pinion 126, rotates counterclockwise, the first rack 128 advances distally toward the end effector 106 and the second rack 130 retracts proximally away from the end effector 106. As the first rack 128 advances distally toward the end effector 106, the first articulation band 132 similarly advances distally toward the end effector 106. As the second rack 130 retracts proximally away from the end effector 106, the second articulation band 136 similarly retracts proximally away from the end effector 106.

In summary, rotation of the articulation control knob 120 enables the first articulation band 132 and the second articulation band 136 to move in a push-and-pull manner. As further described herein, movement of the first articulation band 132 and the second articulation band 136 in the push-and-pull manner enables movement of components at a distal end of the shaft assembly 104, thereby enabling articulation of the closure ring 118 and the end effector 106 relative to the longitudinal axis A-A.

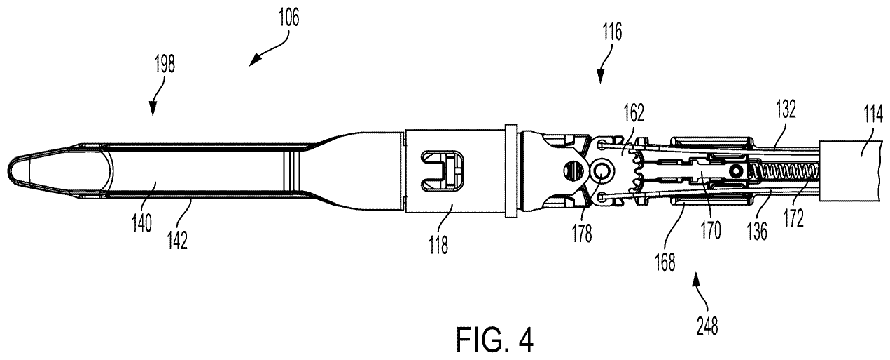

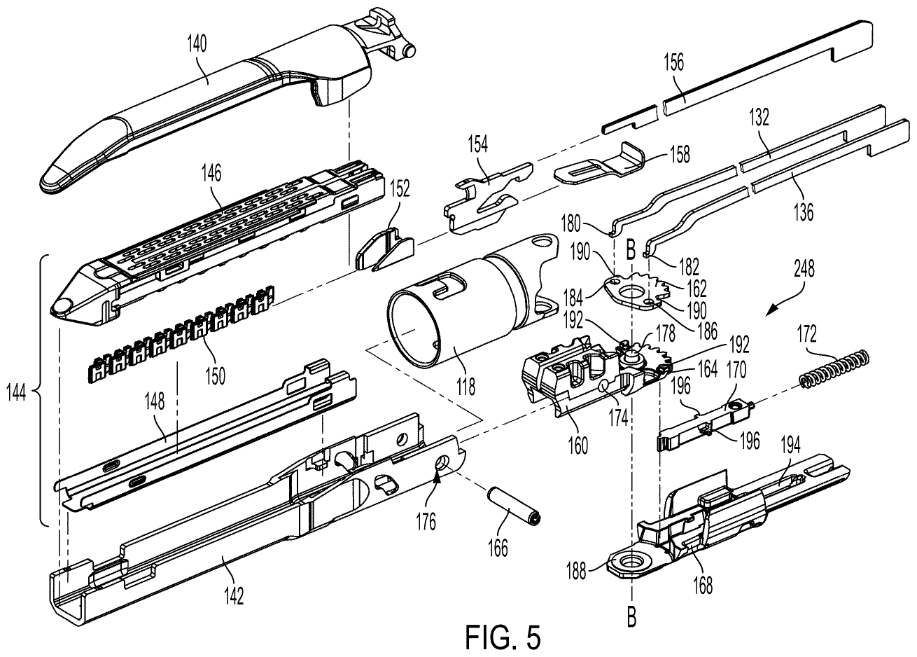

Referring now to FIGS. 4 and 5, the distal end of the shaft assembly 104 and the end effector 106 are disclosed. As shown, the end effector 106 includes an anvil, or a first jaw, 140, an elongate channel, or a second jaw, 142, and a staple cartridge 144. The staple cartridge 144 includes a cartridge body 146, a tray 148, a plurality of staple drivers 150, and a sled 152. In various instances, the staple cartridge 144 is a replaceable staple cartridge, and the tray 148 is removably positioned in the elongate channel 142, and the cartridge body 146, the tray 148, and the plurality of staple drivers 150 are positioned together to form the staple cartridge 144.

The proximal portions of the anvil 140 and the elongate channel 142 are positionable in the closure ring 118, which is coupled to the articulation joint 116. The anvil 140 is movable relative to and toward the elongate channel 142 in response to a distal advancement of the closure ring 118 such that the anvil 140 and the cartridge body 146 of the staple cartridge 144 may clamp tissue therebetween. In various instances, the closure ring 118 may be advanced distally by driving the closure tube 120 distally in response to the closure trigger 110 being actuated. In various instances, the closure trigger 110 being actuated includes the closure trigger 110 pivoting relative to and toward the handle 108.

After the tissue has been clamped between the anvil 140 and the cartridge body 146 of the staple cartridge 144, the tissue may be stapled and cut. Specifically, after the tissue has been clamped, the sled 152 may be advanced distally, which drives the plurality of staple drivers 150 upwards such that the plurality of staple drivers 150 drives a plurality of staples (not shown) through the clamped tissue and against the anvil 140, thereby securing the plurality of staples in the clamped tissue. In various instances, the sled 152 is driven by a knife body 154, which is coupled to and driven by a firing bar 156. In certain instances, the knife body 154 may including camming flanges to further clamp the anvil 140 relative to the elongate channel 142 during a firing stroke. In such instances, the knife body 154 may be referred to as a distal beam head or an I-beam, for example. As the firing bar 156 is advanced distally, the knife body 154 is advanced distally thereby cutting tissue while driving the sled 152. As shown, the sled 152 is positioned distal to the knife body 154 such that the staples are driven and secured to the clamped tissue before the knife body 154 cuts the clamped tissue. In various instances, the firing bar 156 may be advanced distally in response to the firing trigger 112 being actuated. In various instances, the firing trigger 112 being actuated includes the firing trigger 112 pivoting relative to and toward the handle 108.

Referring primarily to FIG. 5, the end effector 106 may include a lockout 158 that is operable with a plate holding body 160 to selectively restrict a distal advancement of the knife body 154 in the absence of an unspent staple cartridge 144 being positioned in the elongate channel 142. Exemplary lockout features are described in, for example, U.S. Pat. No. 9,717,497, titled LOCKOUT FEATURE FOR MOVABLE CUTTING MEMBER OF SURGICAL INSTRUMENT, which issued Aug. 1, 2017; and U.S. Pat. No. 10,335,147, titled METHOD OF USING LOCKOUT FEATURES FOR SURGICAL STAPLER CARTRIDGE, which issued Jul. 2, 2019, each of which is hereby incorporated by reference herein in its respective entirety.

Referring still to FIG. 5, the articulation joint 116 may include a first articulation plate 162, a second articulation plate 164, the plate holding body 160, a channel pin 166, a joint base 168, a lock bar 170, and a spring 172. The first articulation plate 162 and the second articulation plate 164 may include a plurality of gear-like teeth, and corresponding detents positioned therebetween, positioned along a perimeter of the first articulation plate 162 and the second articulation plate 164. The articulation joint 116 is coupled to the end effector 106 via the channel pin 166, which is disposed through an opening 174 formed in the plate holding body 160 and an opening 176 formed in the elongate channel 142. As such, the end effector 106 pivots with the plate holding body 160 about an articulation axis, or pivot axis, B-B defined along a plate holding pin 178, thereby providing articulation of the end effector 106 about the articulation axis B-B, as further described herein. The joint base 168 maintains a fixed position along the longitudinal axis A-A such that the joint base 168 does not pivot about to the articulation axis B-B during an articulation motion, thereby providing a mechanical ground for the articulation joint 116.

As shown, a distal end of the first articulation band 132 includes a first hook 180 and a distal end of the second articulation band 136 includes a second hook 182. The first hook 180 is coupleable with a first plate opening 184 defined in the first articulation plate 162 and the second hook 182 is coupleable with a second plate opening 186 defined in the first articulation plate 162. As a result, a proximal retraction of the first articulation band 132 and a corresponding distal advancement of the second articulation band 136 enables the first articulation plate 162 to rotate clockwise about the articulation axis B-B. A distal advancement of the first articulation band 132 and a corresponding proximal retraction of the second articulation band 136 enables the first articulation plate 162 to rotate counterclockwise about the articulation axis B-B. In various instances, a direction in which the articulation control knob 120 is rotated corresponds to a same direction in which the first articulation plate 162 rotates and, thus, the end effector 106 relative to the shaft assembly 104.

In various instances, the first articulation plate 162 is stacked above the second articulation plate 164. In other instances, the first articulation plate 162 is stacked below the second articulation plate 164. In all instances, the first articulation plate 162 and the second articulation plate 164 are stacked and aligned such that the gear-like teeth of the first articulation plate 162 are aligned with the gear-like teeth of the second articulation plate 164. Similarly, the corresponding detents of the first articulation plate 162 are aligned with the corresponding detents of the second articulation plate 164.

In various instances, the plate holding body 160, the second articulation plate 164, and the plate holding pin 178 are unitary. As a result, rotation of any one component of the plate holding body 160, the second articulation plate 164, or the plate holding pin 178 results in rotation of the other components.

In various instances, the first articulation plate 162 is pivotably coupled to the plate holding pin 178, which is aligned with a joint base opening 188 of the joint base 168 along the pivot axis B-B. As a result, the first articulation plate 162 is pivotable about the articulation axis B-B relative to the plate holding body 160, the second articulation plate 164, the plate holding pin 178, and the joint base 168.

Referring primarily to FIG. 5, the first articulation plate 162 includes cam wings 190 and the second articulation plate 164 includes cam lugs 192 which extend along an axis parallel to the articulation axis B-B and toward the first articulation plate 162. The cam wings 190 and the cam lugs 192 are sized and positioned such that rotation of the first articulation plate 162 enables at least one cam wing 190 to engage at least one cam lug 192, thereby causing the second articulation plate 164 to rotate in a same direction as the first articulation plate 162.

Although the first articulation plate 162 and the second articulation plate 164 are not unitary, and the first articulation plate 162 is enabled to rotate relative to the second articulation plate 164 as described herein, the cam wings 190 and the cam lugs 192 enable the second articulation plate 164 to rotate in response to a rotation of the first articulation plate 162.

In various instances, surgical devices are susceptible to de-articulation motions in which an end effector may move away from the articulated configuration toward an unarticulated configuration due to forces transmitted across an articulation joint. For example, a closure and/or a firing stroke can involve the transmission of significant forces across the articulation joint and into the end effector. Higher forces may be required for thicker and/or denser tissue, for example. It may be desirable to lock an end effector in an articulated configuration to prevent de-articulation of the end effector during a surgical function, such as a jaw closure motion or a staple firing motion, for example.

In various instances, it may be desirable to include a passive articulation lock in which an articulation lock is automatically engaged without a user input. In other instances, it may be desirable to include an active articulation lock in which an articulation lock is engaged in response to a user input. In yet other instances, it may be desirable to include a combination of passive articulation locks and active articulation locks.

Referring primarily to FIGS. 4 and 5, the lock bar 170 meshingly engages the first articulation plate 162 and the second articulation plate 164. Specifically, a distal end of the lock bar 170 meshingly engages the detents defined in the first articulation plate 162 and the second articulation plate 164. The lock bar 170 is distally biased by the spring 172, thereby creating a passive articulation lock 248. The joint base 168 includes a channel, or a seat, 194 for the lock bar 170 to longitudinally translate relative to joint base 168, and the lock bar 170 includes a plurality of bosses 196 sized and positioned to engage a portion of the joint base 168 to restrict a distal advancement of the lock bar 170 beyond a point of contact with the detents defined in the first articulation plate 162 and the second articulation plate 164.

As described herein, the meshing engagement between the lock bar 170 and the detents defined in the first and second articulation plates 162, 164 creates the passive articulation lock 248. In various instances, a biasing force provided distally by the spring 172 to the lock bar 170 is sufficient to maintain an engagement between the distal end of the lock bar 170 and the detents defined in the first and second articulation plates 162, 164. As such, a de-articulation motion during a surgical function is opposed by the contact between the distal end of the lock bar 170 and the respective detents defined in each of the first and second articulation plates 162, 164.

The biasing force provided distally by the spring 172 to the lock bar 170 can be overcome by a user when the user applies an articulation force exceeding a threshold force in order to articulate the end effector 106. For example, if a user provides a rotational force, either clockwise or counterclockwise, to the first articulation plate 162, and thus the second articulation plate 164, via the articulation control knob 120 and the rotational force is sufficient to overcome the biasing force provided distally by the spring 172, the lock bar 170 and the spring 172 will be compressed to permit rotation of the articulation plates 162, 164 about the articulation axis B-B and relative to the distal end of the lock bar 170. After the user rotates the first articulation plate 162, and thus the second articulation plate 164, via the articulation control knob 120, the spring 172 will bias the lock bar 170 distally into a next available gap defined in the first and second articulation plates 162, 164, thereby locking the end effector 106 in a new configuration. In various instances, the passive articulation lock 248 may lock the end effector 106 in the articulated configuration 200. In other instances, however, the passive articulation lock 248 may lock the end effector 106 in an unarticulated configuration 198, as shown in FIG. 4.

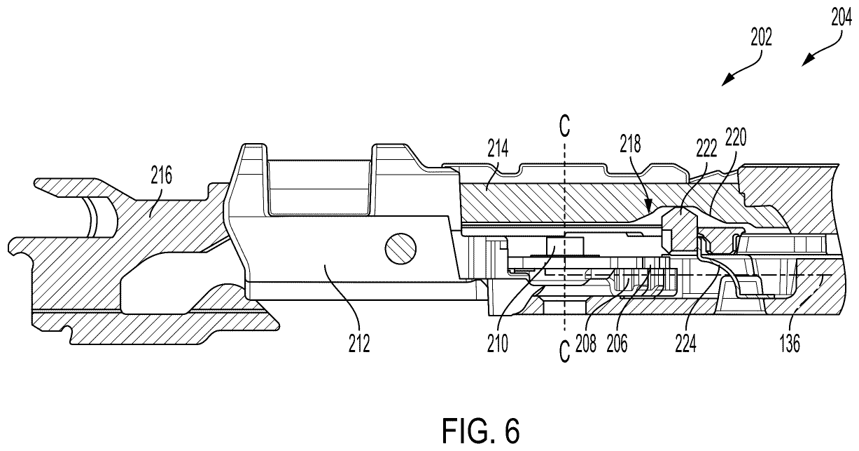

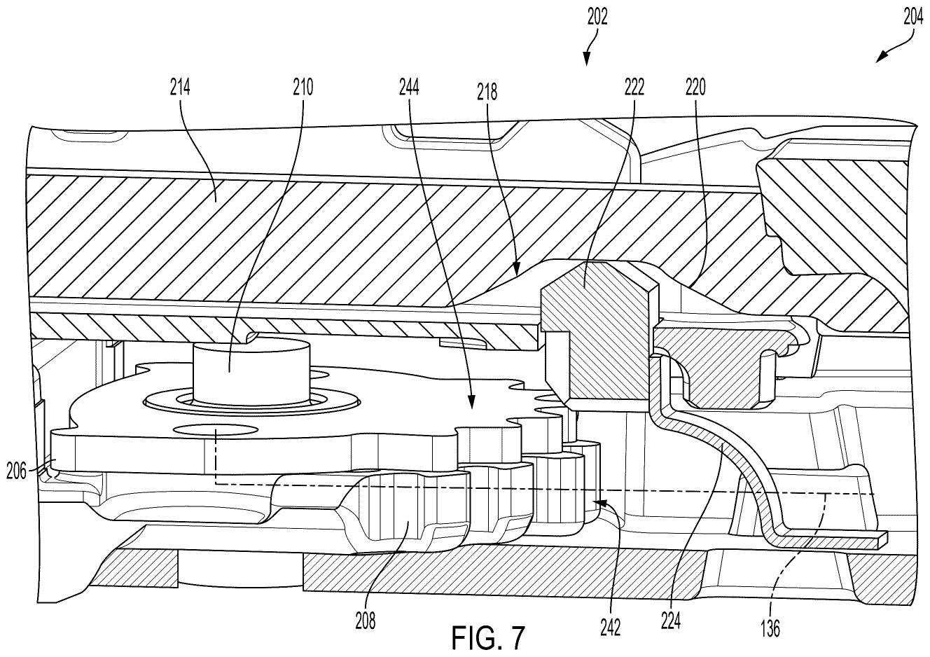

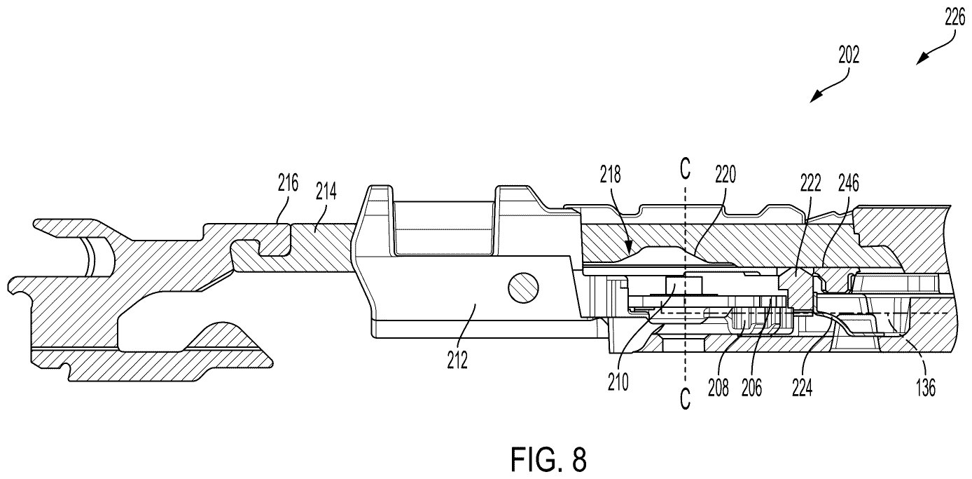

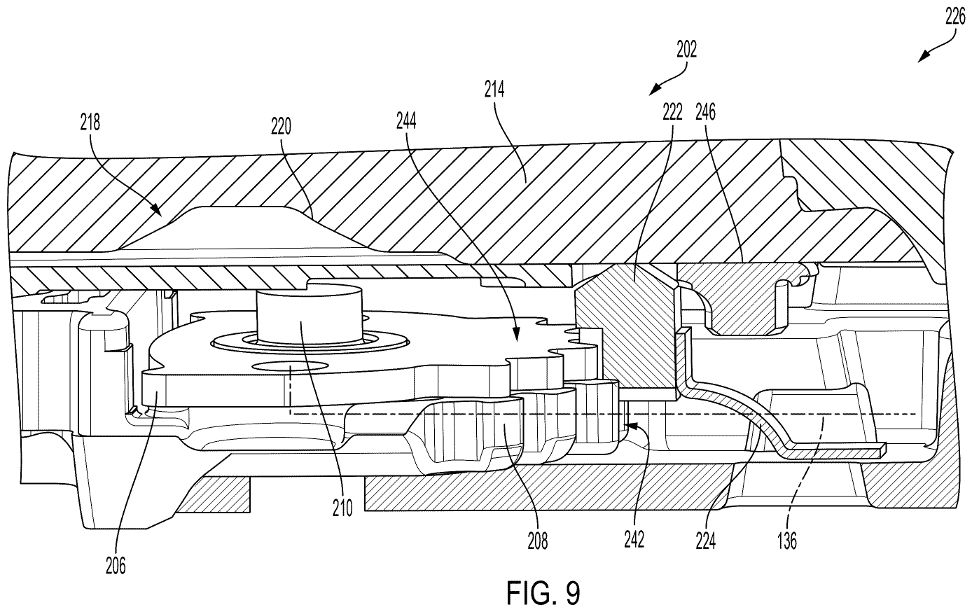

Referring now to FIGS. 6-9, a portion of a shaft assembly including a first active articulation lock 202 is shown. The first active articulation lock 202 is in an unlocked position 204 in FIGS. 6 and 7 and a locked position 226 in FIGS. 8 and 9. The shaft assembly of FIGS. 6-9 is similar in many aspects to the shaft assembly 104 described herein with reference to FIGS. 1-5. The shaft assembly of FIGS. 6-9 and the first active articulation lock 202 thereof are integrated into the shaft assembly 104 of FIGS. 1-5 in various aspects of the present disclosure. For example, the first active articulation lock 202 may be integrated into the shaft assembly 104 of the surgical device 100 in addition to or in lieu of the passive articulation lock 248.

Referring still to FIGS. 6-9, the shaft assembly includes a first articulation plate 206, a second articulation plate 208, a plate holding pin 210 defining a pivot axis C-C, and a plate holding body 212. In various instances, the first articulation plate 206, the second articulation plate 208, the plate holding pin 210, and the plate holding body 212 are substantially similar in structure and function to the first articulation plate 162, the second articulation plate 164, the plate holding pin 178 defining the pivot axis B-B, and the plate holding body 160, respectively.

The shaft assembly further includes a firing bar 214 and a knife body 216 coupled to and driven by the firing bar 214. In various instances, the firing bar 214 and the knife body 216 may be substantially similar to the firing bar 156 and the knife body 154, respectively, described herein. However, unlike the firing bar 156, the firing bar 214 includes a clearance, or recess 218, having at least one cam 220 or camming surface into the firing bar 156 forming at least a portion of the recess 218. As shown, the shaft assembly further includes a locking tab 222 and a spring 224 operable to bias the locking tab 222 toward the firing bar 214.

Referring now to FIGS. 6 and 7, in the unlocked position 204, the locking tab 222 is sized and positioned to be received by the recess 218 defined in the firing bar 214 when the firing bar 214 is in a proximal, home, or proximal-most position. Specifically, the spring 224 biases the locking tab 222 into the recess 218 defined in the firing bar 214. As shown, in the unlocked position 204, the locking tab 222 does not engage the first and second articulation plates 206, 208. Rather, a vertical gap is defined between the locking tab 222 and the first and second articulation plates 206, 208. As a result of the vertical gap defined between the locking tab 222 and the first and second articulation plates 206, 208, the first and second articulation plates 206, 208 and thus the end effector 106 are enabled to rotate about the pivot axis C-C between an unarticulated configuration 198 and an articulated configuration 200 in response to a user input via an articulation control knob, such as the articulation control knob 120, as described herein.

Referring now to FIGS. 8 and 9, as the firing bar 214 is advanced distally, which may be during a staple firing motion in response to the firing trigger 112 being actuated as described herein, the at least one cam 220 moves along the locking tab 222, thereby biasing the locking tab 222 toward the first and second articulation plates 206, 208. For example, a lock-facing surface 246 of the firing bar 214 can hold the locking tab 222 against the bias of the spring 224 and into engagement with the first and second articulation plates 206, 208. It will be understood that the staple firing motion may occur while the first and second articulation plates 206, 208 and thus the end effector 106 are in an unarticulated configuration 198, an articulated configuration 200, or any position therebetween. As the locking tab 222 is biased toward the first and second articulation plates 206, 208, the locking tab 222 meshingly engages a slot 242 defined between a plurality of gear-like teeth and along a perimeter 244 of the first and second articulation plates 206, 208. The teeth can define locked articulation indexes defining articulation angles (e.g. 5-degree, 10-degree, 15-degree, 30-degree angle intervals at which the articulation angle of the end effector 106 can be set. As the at least one cam 220 distally advances past the locking tab 222, the meshing engagement between the locking tab 222 and the slot 242 is maintained during actuation, or distal advancement, of the firing bar 214 via the lock-facing surface 246 of the firing bar 214. As shown, the lock-facing surface 246 continues to bias the locking tab 222 toward the first and second locking plates 206, 208 during actuation, or distal advancement, of the firing bar 214.

Referring primarily still to FIGS. 8 and 9, the meshing, or locking, engagement between the locking tab 222 and the slot 242 forms the first active articulation lock 202, thereby preventing the first and second articulation plates 206, 208 from rotating about the articulation axis C-C and, thus, preventing articulation of the end effector 106 relative to the shaft assembly 104 and the longitudinal axis A-A defined by the shaft assembly 104.

In various instances, it may be desirable to integrate both the passive articulation lock 248 and the first active articulation lock 202 into a surgical device, such as the surgical device 100. In such instances, the passive articulation lock 248 and the first active articulation lock 202 may be laterally offset from one another such that both the lock bar 170 and the locking tab 222 may meshingly, or lockingly, engage the first and second articulation plates 206, 208. In such instances, the lock bar 170 may meshingly engage the first and second articulation plates 206, 208 via a first slot while the locking tab 222 may meshingly engage the first and second articulation plates 206, 208 via a second, different slot. It will be understood that additional configurations are contemplated herein to integrate both the passive articulation lock 248 and the first active articulation lock 202 into a surgical device, such as the surgical device 100.

In various instances, the first active articulation lock 202 is a self-locking, or self-actuated, articulation lock based on an actuation of the staple firing motion described herein. In other words, the staple firing motion described herein may automatically move the first active articulation lock 202 into the locked position 226 by camming the locking tab 222 during the staple firing motion to a locking engagement with the slot 242, thereby preventing rotation of the first and second articulation plates 206, 208 about the pivot axis C-C and, thus, preventing articulation of the end effector 106 relative to the longitudinal axis A-A. In various instances, the first active articulation lock 202 may automatically return to the unlocked position 204 upon retraction of the firing bar 214. As the firing bar 214 is retracted proximally toward the proximal-most position, the recess 218 may be re-aligned with the locking tab 222 such that the spring 224 may vertically bias the locking tab 222 away from the slot 242 and into the recess 218, thereby returning the first active articulation lock 202 to the unlocked position 204.

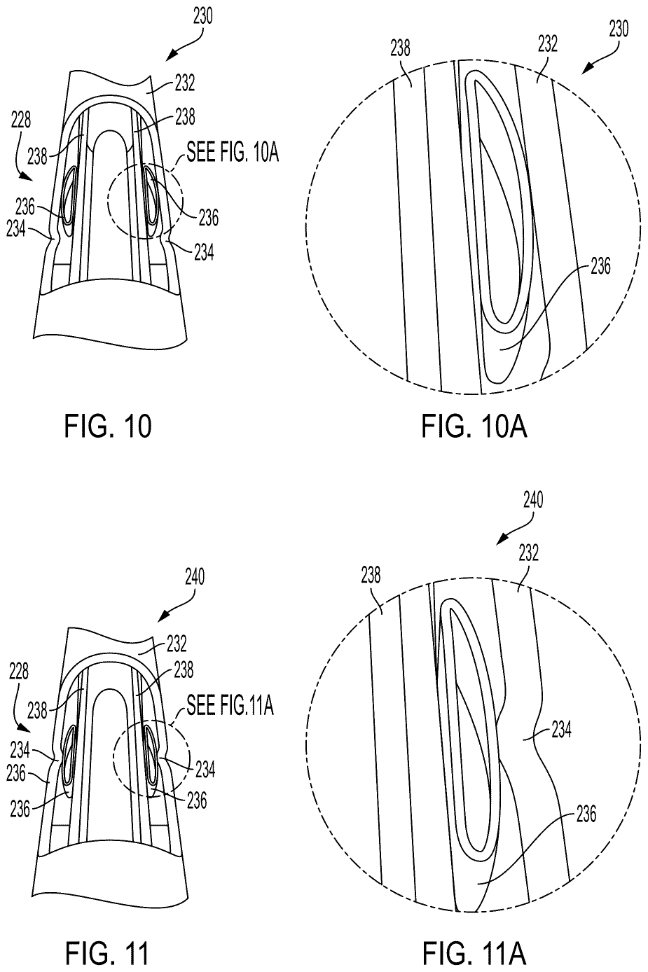

Referring now to FIGS. 10 and 11, a portion of a shaft assembly including a second active articulation lock 228 in an unlocked position 230 is disclosed in FIGS. 10 and 10A, and a portion of the shaft assembly including the second active articulation lock 228 in a locked position 240 is disclosed in FIGS. 11 and 11A. The shaft assembly of FIGS. 10 and 11 is substantially similar to the shaft assembly 104 described herein with reference to FIGS. 1-5, and the following aspects of the shaft assembly of FIGS. 10 and 11 may be integrated into the shaft assembly 104 of FIGS. 1-5. Specifically, the second active articulation lock 228 may be integrated into the shaft assembly 104 of the surgical device 100 in addition to or in lieu of the passive articulation lock 248.

Referring still to FIGS. 10 and 11, the shaft assembly includes a closure tube 232 and articulation bands 238. In various instances, the closure tube 232 and the articulation bands 238 may be substantially similar to the closure tube 114 and the first and second articulation bands 132, 136, respectively, described herein, thereby enabling the shaft assembly of FIGS. 10 and 11 to perform substantially similar surgical functions to the shaft assembly 104 of FIGS. 1-5.

The shaft assembly further includes a protrusion 234 extending laterally inward toward a compressible clamp, or spring, 236 positioned laterally between the protrusion 234 and the articulation bands 238. The protrusion 234 may be defined in, or on, the closure tube 232. In various instances, the protrusion 234 may be a stamped indent and integrally formed with the actuator (e.g. the closure tube 232). In other instances, the protrusion 234 may be a separate component coupled to an inner surface of the closure tube 132 such that the another component extends laterally inward toward the compressible clamp 236. For example, the separate component can be welded and/or snapped into the closure tube 232.

Referring now to FIGS. 10 and 10A, in the unlocked position 230, the protrusion 234 is positionable proximal to the compressible clamp 236 such that the protrusion 234 does not engage the compressible clamp 236. In other words, in the unlocked position 230, a gap is defined between the protrusion 234 and the compressible clamp 236. As a result, the articulation bands 238 are enabled to translate along the longitudinal axis A-A in response to a user input via an articulation control knob, such as the articulation control knob 120, as described herein, thereby causing rotation of the end effector 106 relative to the longitudinal axis A-A.

Referring now to FIGS. 11 and 11A, in the locked position 240, the closure tube 232 is advanced distally, which may be during jaw closure in response to the closure trigger 110 being actuated as described herein, and the protrusion 234 engages the compressible clamp 236. As the closure tube 232 is advanced distally, the protrusion 234 engages the compressible clamp 236, thereby applying a compressive force to the compressible clamp 236 and thus the articulation bands 238. As a result of the compressive force applied to the compressible clamp 236 and, thus, the articulation bands 238, a frictional force between the compressible clamp 236 and the articulation bands 238 is increased, thereby forming the second active articulation lock 228 and preventing translation of the articulation bands 238 along the longitudinal axis A-A.

The compressible clamp 236 can be a spring, such as a leaf spring, for example. In certain instances, the compressible clamp 236 can be referred to as an elastomeric or resilient clamp. The compressible clamp 236 may be a stamped or molded spring, for example. The compressible clamp 236 defines a hollow, semi-circular or semi-elliptical profile; however, alternative geometries are contemplated. In various aspects, the profile of the compressible clamp 236 can be ramped in the directions of closure tube 232 motion (e.g. in the proximal and distal directions) to prevent snagging between the closure tube 232 and the compressible clamp 236. In various instances, the compressible clamp 236 can include a texture or grip on at least the surface of the compressible clamp 236 facing the articulation bands 238 to increase the coefficient of friction between the compressible clamp 236 and the articulation bands 238.

It will be understood that the second active articulation lock 228 may be positionable at any portion of the closure tube 232 along a length of the shaft assembly. In various instances, positioning the second active articulation lock 228 at a distal end of the closure tube 232 and adjacent to the articulation joint can maximize stability of the end effector 106 and reduces de-articulation of the end effector 106 during a surgical function.

In various instances, the second active articulation lock 228 is a self-locking, or self-actuated, articulation lock based on an actuation of the closing motion, or jaw closure, described herein. In other words, the closing motion described herein may automatically move the second active articulation lock 228 from an unlocked position 230 to a locked position 240. As the closure tube 232, and thus the protrusion 234, is advanced distally from a first position corresponding to the unlocked position 230 to a second position corresponding to the locked position 240, the protrusion 234 may automatically engage the compressible clamp 236, thereby moving the second active articulation lock 228 into the locked position 240 in which a frictional force between the compressible clamp 236 and the articulation bands 238 is increased and translation of the articulation bands 238 along the longitudinal axis A-A is prevented. In various instances, the second active articulation lock 228 may automatically return to the unlocked position 230 upon retraction of the closure tube 232. As the closure tube 232 is retracted proximally toward the first position, the protrusion 234 is similarly retracted proximally toward the first position and the protrusion 234 is no longer positioned to engage the compressible clamp 236. As the locking contact between the protrusion 234 and the compressible clamp 236 is released, the second active articulation lock 228 automatically returns to the unlocked position 230.

As described herein, it may be desirable to include a combination of passive articulation locks and/or active articulation locks. In various instances, a combination of passive articulation locks and/or active articulation locks may include a combination of the passive articulation lock 248 with the first active articulation lock 202. In other instances, a combination of passive articulation locks and/or active articulation locks may include a combination of the passive articulation lock 248 with the second active articulation lock 228. In yet other instances, a combination of passive articulation locks and/or active articulation locks may include any suitable passive articulation lock in combination with one of the first active articulation lock 202 or the second active articulation lock 228.

In various instances, regardless of the combination of passive articulation locks and/or active articulation locks, both the passive articulation lock and the active articulation lock may be operable to resist de-articulation forces. In various instances, the passive articulation lock may be operable to resist de-articulation forces up to a first threshold, and the active articulation lock may be operable to resist de-articulation forces up to a second threshold. In various instances, the first and second thresholds may be different thresholds. For example, the second threshold may be greater than the first threshold. In other instances, the first and second thresholds may be the same.

It is contemplated that the articulation locks described herein can also be incorporated into non-stapling, articulatable surgical tools, such as surgical graspers, cutters, energy devices (e.g., ultrasonic, radio-frequency (RF), etc.), imaging devices, access devices, and/or fluid delivery and/or extraction devices (e.g., irrigators, smoke evacuators, etc.), for example. Various features of the articulation locks described herein can also be incorporated into robotic surgical tools.

Examples of the devices, systems, and methods disclosed herein are provided in the following numbered clauses. An aspect of the devices, systems, and methods may include any one, more than one, and any combination of the numbered clauses below:
Clause 1: A surgical device, comprising: an elongate shaft; an end effector; an articulation joint, wherein the end effector is rotatable relative to the elongate shaft at the articulation joint through an articulation motion between an unarticulated configuration and an articulated configuration; an articulation band extending distally to the articulation joint, wherein the articulation band is translatable to rotate the end effector through the articulation motion; an actuator actuatable to transmit a force across the articulation joint to the end effector to effect a surgical function; and an articulation lock structured to lock the articulation joint in the articulated configuration, wherein the articulation lock is movable between an unlocked position and a locked position, and wherein an actuation of the actuator is structured and positioned to move the articulation lock from the unlocked position to the locked position.
Clause 2: The surgical device of Clause 1, wherein the surgical function comprises staple firing.
Clause 3: The surgical device of Clause 1, wherein the articulation lock comprises an articulation plate and a locking tab, wherein the articulation plate is rotatable about an axis to rotate the end effector through the articulation motion based on translation of the articulation band, wherein the articulation plate comprises at least one slot positioned along a perimeter of the articulation plate, and wherein, in the locked position, the locking tab is positionable in the at least one slot to prevent rotation of the end effector relative to the elongate shaft.
Clause 4: The surgical device of Clause 3, wherein the articulation lock further comprises a spring to bias the locking tab toward the unlocked position.
Clause 5: The surgical device of Clause 3, wherein the actuator comprises a firing bar, wherein the firing bar comprises a recess, and wherein, in a first position of the firing bar corresponding to the unlocked position of the articulation lock, the recess is sized and positioned to receive the locking tab.
Clause 6: The surgical device of Clause 5, wherein the recess comprises at least one cam, and wherein an actuation of the firing bar moves the at least one cam along the locking tab to cam the locking tab into locking engagement with the at least one slot.
Clause 7: The surgical device of Clause 6, wherein, after the at least one cam cams the locking tab into locking engagement with the at least one slot, a surface of the firing bar maintains the locking tab in the locked position during actuation of the firing bar.
Clause 8: The surgical device of Clause 3, wherein the locked position is defined by a meshing engagement between the articulation plate and the locking tab, and wherein the unlocked position is defined by a gap between the articulation plate and the locking tab.
Clause 9: The surgical device of Clause 3, wherein the articulation plate comprises a first articulation plate and a second articulation plate, wherein the first articulation plate is coupled to the articulation band, wherein the second articulation plate is coupled to the end effector, wherein the first articulation plate is rotatably coupled to the second articulation plate (208), and wherein the locking tab meshingly engages the first articulation plate and the second articulation plate in the locked position of the articulation lock.
Clause 10: The surgical device of Clause 1, wherein the surgical function comprises jaw closure.
Clause 11: The surgical device of Clause 1, wherein the actuator comprises a closure tube, wherein the articulation lock comprises a spring positioned laterally between the closure tube and the articulation band, wherein the closure tube comprises a protrusion extending laterally inward toward the spring, and wherein, in the locked position, the protrusion is positionable to apply a compressive force to the spring.
Clause 12: The surgical device of Clause 11, wherein the protrusion comprises a stamped indent in the closure tube.
Clause 13: The surgical device of Clause 11, wherein the locked position is defined by a locking contact between the protrusion and the spring, and wherein the unlocked position is defined by a gap between the protrusion and the spring.
Clause 14: The surgical device of Clause 11, wherein, in the locked position, a frictional force between the spring and the articulation band prevents translation of the articulation band, thereby preventing rotation of the end effector through the articulation motion.
Clause 15: The surgical device of Clause 1, wherein the articulation lock is a secondary articulation lock, and wherein the surgical device further comprises a primary articulation lock.
Clause 16: The surgical device of Clause 15, wherein the primary articulation lock is a passive articulation lock to resist de-articulation forces up to a first threshold, wherein the secondary articulation lock is an active articulation lock to resist de-articulation forces up to a second threshold, and wherein the second threshold is greater than the first threshold.
Clause 17: The surgical device of Clause 1, wherein the articulation band comprises a plurality of articulation bands to push and pull the articulation joint through the articulation motion.
Clause 18: The surgical device of Clause 1, wherein the actuator comprises at least one of a closure trigger or a firing trigger.
Clause 19: A surgical device, comprising: an elongate shaft; an end effector; an articulation joint, wherein the end effector is rotatable relative to the elongate shaft at the articulation joint through an articulation motion between an unarticulated configuration and an articulated configuration; an actuator actuatable to transmit a force to the end effector to effect a surgical function; and an articulation lock structured to lock the articulation joint, wherein the articulation lock is movable between an unlocked position and a locked position, and wherein an actuation of the actuator is structured and positioned to move the articulation lock from the unlocked position to the locked position.
Clause 20: A surgical device, comprising: an end effector; an articulation joint, wherein the end effector is articulatable about an axis of the articulation joint between an unarticulated configuration and an articulated configuration; an actuator actuatable to transmit a force to the end effector to effect a surgical function; and an articulation lock structured to lock the articulation joint, wherein the articulation lock is movable between an unlocked position and a locked position, and wherein an actuation of the actuator is structured and positioned to move the articulation lock from the unlocked position to the locked position.

The terms “proximal” and “distal” are used herein with reference to a clinician manipulating a surgical device or, in the case or a robotic tool, to a robot manipulating the tool. The term “proximal” refers to the portion closest to the clinician or the robot (e.g., the housing) and the term “distal” refers to the portion located away from the clinician or robot (e.g., the end effector). It will be further appreciated that, for convenience and clarity, spatial terms such as “vertical”, “horizontal”, “up”, “down”, “clockwise”, and “counterclockwise” may be used herein with respect to the drawings. However, surgical devices are used in many orientations and positions, and these terms are not intended to be limiting and/or absolute.

The singular forms “a,” “an,” and “the” include plural referents unless the context clearly dictates otherwise. Furthermore, the use of “couple”, “coupled”, or similar phrases should not be construed as being limited to a certain number of components or a particular order of components unless the context clearly dictates otherwise.

As used herein, the terms “about” or “approximately” for any numerical values or ranges indicate a suitable dimensional tolerance (e.g., ±20%) that allows the part or collection of components to function for its intended purpose as described herein.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flow diagrams are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures or characteristics may be combined in any suitable manner in one or more aspects.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material. None is admitted to be prior art.

In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

1-15. (canceled)

16. A surgical device, comprising:
an elongate shaft;
an end effector;
an articulation joint, wherein the end effector is rotatable relative to the elongate shaft at the articulation joint through an articulation motion between an unarticulated configuration and an articulated configuration;
an articulation band extending distally to the articulation joint, wherein the articulation band is translatable to rotate the end effector through the articulation motion;
an actuator actuatable to transmit a force across the articulation joint to the end effector to effect a surgical function; and
an articulation lock structured to lock the articulation joint in the articulated configuration, wherein the articulation lock is movable between an unlocked position and a locked position, and wherein an actuation of the actuator is structured and positioned to move the articulation lock from the unlocked position to the locked position.

17. The surgical device of claim 16, wherein the surgical function comprises staple firing.

18. The surgical device of claim 16, wherein the articulation lock comprises an articulation plate and a locking tab, wherein the articulation plate is rotatable about an axis to rotate the end effector through the articulation motion based on translation of the articulation band, wherein the articulation plate comprises at least one slot positioned along a perimeter of the articulation plate, and wherein, in the locked position, the locking tab is positionable in the at least one slot to prevent rotation of the end effector relative to the elongate shaft.

19. The surgical device of claim 18, wherein the articulation lock further comprises a spring to bias the locking tab toward the unlocked position.

20. The surgical device of claim 18, wherein the actuator comprises a firing bar, wherein the firing bar comprises a recess, and wherein, in a first position of the firing bar corresponding to the unlocked position of the articulation lock, the recess is sized and positioned to receive the locking tab.

21. The surgical device of claim 20, wherein the recess comprises at least one cam, and wherein an actuation of the firing bar moves the at least one cam along the locking tab to cam the locking tab into locking engagement with the at least one slot.

22. The surgical device of claim 21, wherein, after the at least one cam cams the locking tab into locking engagement with the at least one slot, a surface of the firing bar maintains the locking tab in the locked position during actuation of the firing bar.

23. The surgical device of claim 18, wherein the locked position is defined by a meshing engagement between the articulation plate and the locking tab, and wherein the unlocked position is defined by a gap between the articulation plate and the locking tab.

24. The surgical device of claim 18, wherein the articulation plate comprises a first articulation plate and a second articulation plate, wherein the first articulation plate is coupled to the articulation band, wherein the second articulation plate is coupled to the end effector, wherein the first articulation plate is rotatably coupled to the second articulation plate, and wherein the locking tab meshingly engages the first articulation plate and the second articulation plate in the locked position of the articulation lock.

25. The surgical device of claim 16, wherein the surgical function comprises jaw closure.

26. The surgical device of claim 16, wherein the actuator comprises a closure tube, wherein the articulation lock comprises a spring positioned laterally between the closure tube and the articulation band, wherein the closure tube comprises a protrusion extending laterally inward toward the spring, and wherein, in the locked position, the protrusion is positionable to apply a compressive force to the spring.

27. The surgical device of claim 26, wherein the protrusion comprises a stamped indent in the closure tube.

28. The surgical device of claim 26, wherein the locked position is defined by a locking contact between the protrusion and the spring, and wherein the unlocked position is defined by a gap between the protrusion and the spring.

29. The surgical device of claim 26, wherein, in the locked position, a frictional force between the spring and the articulation band prevents translation of the articulation band, thereby preventing rotation of the end effector through the articulation motion.

30. The surgical device of claim 16, wherein the articulation lock is a secondary articulation lock, and wherein the surgical device further comprises a primary articulation lock.

31. The surgical device of claim 30, wherein the primary articulation lock is a passive articulation lock to resist de-articulation forces up to a first threshold, wherein the secondary articulation lock is an active articulation lock to resist de-articulation forces up to a second threshold, and wherein the second threshold is greater than the first threshold.

32. The surgical device of claim 16, wherein the articulation band comprises a plurality of articulation bands to push and pull the articulation joint through the articulation motion.

33. The surgical device of claim 16, wherein the actuator comprises at least one of a closure trigger or a firing trigger.

34. A surgical device, comprising:
an elongate shaft;
an end effector;
an articulation joint, wherein the end effector is rotatable relative to the elongate shaft at the articulation joint through an articulation motion between an unarticulated configuration and an articulated configuration;
an actuator actuatable to transmit a force to the end effector to effect a surgical function; and
an articulation lock structured to lock the articulation joint, wherein the articulation lock is movable between an unlocked position and a locked position, and wherein an actuation of the actuator is structured and positioned to move the articulation lock from the unlocked position to the locked position.

35. A surgical device, comprising: |

an end effector;
an articulation joint, wherein the end effector is articulatable about an axis of the articulation joint between an unarticulated configuration and an articulated configuration;
an actuator actuatable to transmit a force to the end effector to effect a surgical function; and
an articulation lock structured to lock the articulation joint, wherein the articulation lock is movable between an unlocked position and a locked position, and wherein an actuation of the actuator is structured and positioned to move the articulation lock from the unlocked position to the locked position.
